(19) United States
(12) Patent Application Publication
(10) Pub. No.: US 2007/0011794 A1
(43) Pub. Date: Jan. 18, 2007

(54) Assembled safety cap

(76) Inventor: Chieh-Lun Wang Lee, Yung Kang City (TW)

Correspondence Address: NIKOLAI & MERSEREAU, P.A., 900 SECOND AVENUE SOUTH, SUITE 820, MINNEAPOLIS, MN, 55402, US

(21) Appl. No.: 11/183,623

(22) Filed: Jul. 18, 2005

(51) Int. Cl. A61F 9/00

(52) U.S. Cl. 2/10

(57) ABSTRACT

A safety cap includes a crown having two engagements respectively formed on two opposed sides of the crown and each engagement having an extension hole defined therein and a peak detachably connected to the crown and having two extensions respectively formed on two opposed sides of the peak to correspond to the two extension holes such that the two extensions are interference-fitted into the extension holes of the two engagements to secure engagement between the crown and the peak.

### BACKGROUND OF THE INVENTION

1. Field of the Invention

The present invention relates to a safety cap and more particularly to an assembled safety cap having a peak detachably connected to a crown of the safety cap to allow the user to attach/detach the peak to/from the crown.

2. Description of Related Art

In construction sites or hazardous sites, workers and visitors are required to wear safety cap to prevent head injuries arising from falling objects. However, the conventional safety cap having a peak integrally formed on a side face of the crown blocks the sight of the workers such that workers are not able to monitor whether there is potential or immediate danger to their lives in that the peak is made of a material that is not transparent. In order to obviate the potential danger to the worker's life, a new kind of safety cap (30), as shown in FIG. 3, is introduced to the market and has a transparent peak (31) integrally formed with the crown of the safety cap (30) so that the worker wearing this improved safety cap is able to monitor via the transparent peak if there are potential falling objects. Still, this improved safety cap suffers from the drawback that the transparent peak blurs due to abrasive contact with objects, which hinders the worker's sight.

To overcome the shortcomings, the present invention tends to provide an improved safety cap to mitigate the aforementioned problems.

### SUMMARY OF THE INVENTION

The primary objective of the present invention is to provide an improved safety cap having a transparent peak detachably connected to the crown of the safety cap.

In one aspect of the present invention, the crown of the safety cap has two engagements respectively formed on two opposed sides of the crown and each engagement has an extension hole defined therein and a groove defined in an inner face defining the extension hole. The peak has two extensions respectively formed on two opposed sides of the peak and each extension has a hook formed on a free end thereof to correspond to the groove in a corresponding one of the extension holes of the engagements such that the peak is able to be detachably connected to the crown.

Other objects, advantages and novel features of the invention will become more apparent from the following detailed description when taken in conjunction with the accompanying drawings.

### BRIEF DESCRIPTION OF THE DRAWINGS

FIG. 1 is an exploded perspective view of the safety cap of the present invention;

FIG. 2 is a perspective view showing that the safety cap is assembled; and

FIG. 3 is a perspective view of a conventional safety cap.

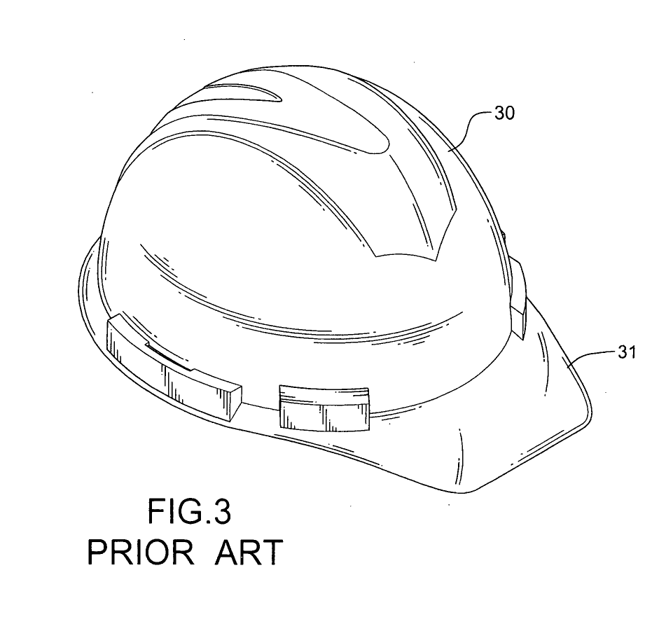

### DETAILED DESCRIPTION OF THE PREFERRED EMBODIMENT

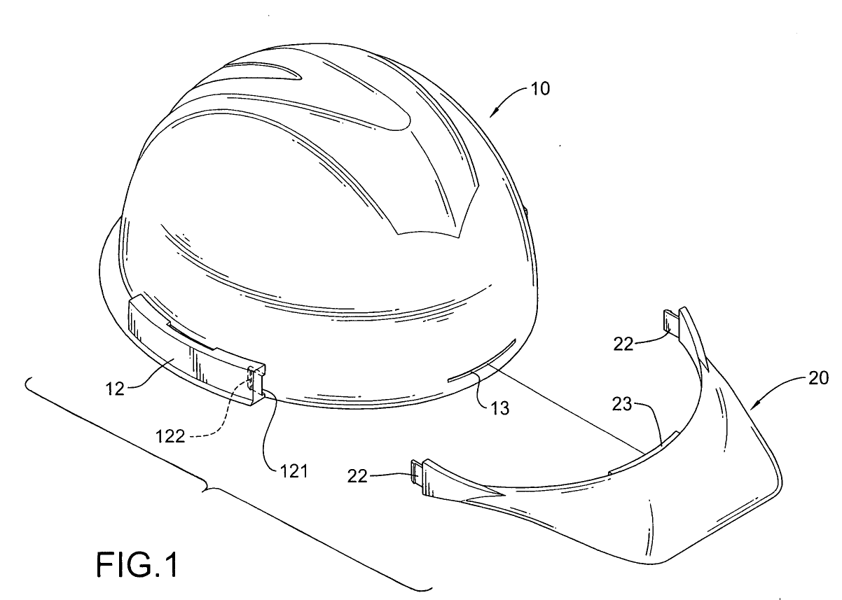

With reference to FIG. 1, it is noted that the safety cap in accordance with the present invention includes a crown (10) and a peak (20).

The crown (10) has two engagements (12) respectively formed on two opposed sides of the crown (10) and each engagement (12) has an extension hole (121) defined therein and a groove (122) defined in an inner side face defining the extension hole (121). A slit (13) is defined in the crown (10) and located between the two engagements (12).

The peak (20) has two extensions (21) respectively stretching from two opposed sides of the peak (20) to correspond to the two extension holes (121) of the engagements (12). Each extension (21) has a hook (22) formed on a free end of the extension (21) to correspond to the grooves (122) in the two extension holes (121). Further, an insert (23) is formed on a peripheral edge of the peak (20) to correspond to the slit (13) of the crown (10).

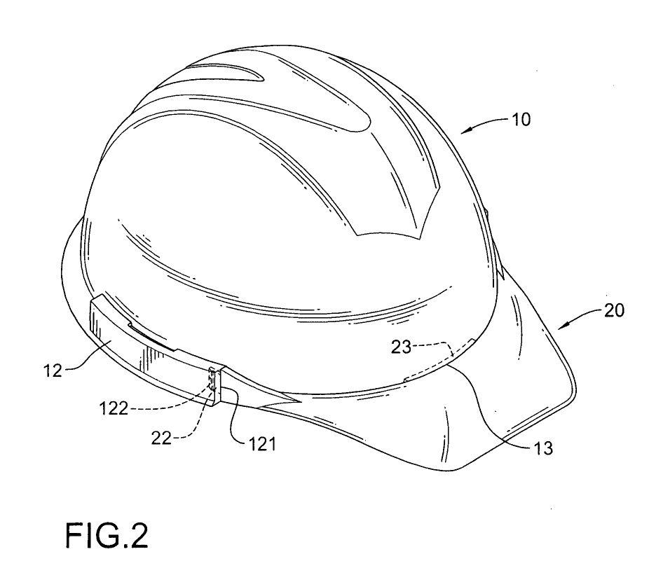

With reference to FIG. 2, it is noted that when the peak (20) is to be connected to the crown (10), each extension (21) is extended into a corresponding one of the two extension holes (121) and then the hook (122) is able to be received in the groove (122). Further, the insert (23) is inserted into the slit (13) to prevent the peak (20) from rocking relative to the crown (10).

It is noted from the aforementioned description that the peak (20) is made of a transparent material and is detachably connected to the crown (10) such that the user of the safety cap is able to detach the peak (20) from the crown (10) as required to prevent unnecessary abrasion to the peak (20). Thus the life span of the peak (20) is prolonged.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A safety cap comprising:
a crown having two engagements respectively formed on two opposed sides of the crown and each engagement having an extension hole defined therein; and
a peak detachably connected to the crown and having two extensions respectively formed on two opposed sides of the peak to correspond to the two extension holes such that the two extensions are interference-fitted into the extension holes of the two engagements to secure engagement between the crown and the peak.

2. The safety cap as claimed in claim 1, wherein each engagement has a groove defined in an inner side face defining the extension hole and each extension has a hook formed on a distal end of the extension to correspond to the groove so that after the extension is extended into the corresponding extension hole, the hook is securely received in the groove to secure the engagement between the crown and the peak.

3. The safety cap as claimed in claim 2 further comprising a slit defined in the crown and being located between the two engagements and an insert formed on the peak and between the two extensions and extended into the slit to prevent rocking of the peak relative to the crown.
